(19) United States
(12) Patent Application Publication
(10) Pub. No.: US 2023/0178175 A1
(43) Pub. Date: Jun. 8, 2023

(54) CANCER RISKS PREDICTION METHOD AND SYSTEM BASED ON BIOLOGICAL AGE USING FAMILY HISTORY AND LIFESTYLES INFORMATION

(71) Applicant: MEDIAGE Co.,Ltd, Seongnam-si (KR)

(72) Inventor: Chul Young BAE, Anyang-si (KR)

(21) Appl. No.: 17/752,803

(22) Filed: May 24, 2022

(30) Foreign Application Priority Data
Dec. 3, 2021 (KR) 10-2021-0171964
May 6, 2022 (KR) 10-2022-0056033

(51) Int. Cl. G16B 20/00; G16H 50/30; G16H 10/20

(52) U.S. Cl. CPC G16B 20/00; G16H 50/30; G16H 10/20

(57) ABSTRACT

A method and system for predicting a risk of individual cancer incidence based on biological age obtained by reflecting family history and lifestyle according to the present disclosure) includes an input unit configured to receive, when basic information such as gender/age and biomarker information such as a medical check-up result and questionnaire information of a customer are provided, the basic information, biomarker information, and questionnaire information of the customer, a biological age measurement unit configured to measure biological age obtained by reflecting family history and lifestyle using the received information, a cancer incidence risk prediction unit configured to predict a risk of incidence for approximately 20 types of individual cancers based on the biological age, an analysis result generation unit configured to generate result report information, a content generation unit, and a service server configured to provide the result report information to a customer, and approximately 20 types of individual cancer.

### CROSS-REFERENCE TO RELATED APPLICATION

This application claims priority to Korean Patent Application No. 10-2021-0171964 filed on Dec. 3, 2021, Korean Patent Application No. 10-2022-0056033 filed on May 6, 2022 and all the benefits accruing therefrom under 35 U.S.C. § 119, the contents of which are incorporated by reference in their entirety.

### BACKGROUND

The present disclosure relates to a method and system for predicting the risk of incidence for individual cancers based on biological age obtained by reflecting a family history and lifestyle.

Humans go through a biological process in which physical and cognitive functions decrease with increasing age after being born and growing for a certain period of time. This phenomenon is called aging. As such, although resident registration age is generally used as an indicator of aging, it is difficult to explain that resident registration age is an optimal indicator of aging progress due to reasons such as genotype, lifestyle, and environmental diversity, in light of the possibility that a 50-year-old individual may have the body functions of a 60-year-old and a 60-year-old individual may have the body functions of a 50-year-old.

In 1988, a method of measuring biological age and using it as a cornerstone for anti-aging was disclosed in ‘Biological Aging Measurement’ by Dr. Widin, and the cornerstone of biological age was prepared. Biological age, which is a new indicator that overcomes the limitations of aging explanation given by resident registration age and can explain relative aging by using biomarkers that change according to age in consideration of the individual's health condition.

Meanwhile, cancer is a disease in which cells exhibit abnormal expression due to abnormalities in the process of cell growth and death. Although an exact cause of cancer is not known, cancer is affected by lifestyle and environmental exposure, and shows a rapid increase in the incidence of cancer due to genetic variation caused by aging. Considering that 59% of all cancers in men and 52% of all cancers in women belong to an age group of 65 years or older in the survey of cancer incidence and survival time in the elderly over 65 years of age, it can be seen that more than half of all cancers occur after 65 years of age.

Thus, aging can be regarded as the most important risk factor for cancer. As described above, biological age is more suitable for explaining aging than resident registration age. Therefore, when reflecting major lifestyles such as the family history, smoking, drinking, and exercise, which are risk factors that increase cancer incidence, in the biological age and predicting the risk of cancer incidence based on this, a more accurate prediction is possible, as well as the difference in the risk of cancer incidence depending on the presence or absence of each risk factor.

In relation to biological age, the following PTL 1 (Korea Patent No. 10-1328643) provides an apparatus and Method of predicting biological age, but a Method of predicting the risk of cancer incidence using this is not known.

### SUMMARY

The present disclosure provides a method and system for providing a relative risk of cancer incidence compared to the same age group by measuring the biological age, which is actual age of the body compared to the resident registration age, using biomarkers that change according to age, and correcting the biological age by using the family history and lifestyle as variables for the calculated biological age.

In accordance with an exemplary embodiment, there is provided a system for calculating a risk of cancer incidence based on biological age, which is applied to a system for predicting a risk of individual cancer incidence, including an input unit configured to receive basic information such as gender and age, biomarker information including a medical check-up result, and questionnaire information of a subject, an analysis unit that comprises a biological age measurement unit configured to calculate biological age of the subject based on the basic information and the biomarker information of the subject, and a cancer incidence risk prediction unit configured to predict a risk of incidence for individual cancers based on the biological age of the subject.

In the system, the biomarker information may include at least one or more of waist-hip ratio (WHR), creatinine clearance rate (CCr), creatinine (Cr), forced expiratory volume in one second (FEV1), glycated hemoglobin (HbA1c), albumin globulin ratio (AGR), blood urea nitrogen (BUN), waist circumference (WC), total protein (TP), Amylase, homocysteine (HOMO), ALT (GPT), fasting blood sugar (FBS), systolic blood pressure (SBP), CPK, forced vital capacity (FVC), AST (GOT), gamma GTP (GGTP), urine specific gravity (USG), body fat % (BF %), body mass index (BMI), ALP, direct bilirubin (DB), albumin (ALB), lean body mass % (LBM %), total cholesterol (TC), and triglyceride (TG), biological age measurement unit may be configured to calculate basal biological age according to Calculation formula 1 below when the subject is a male and calculate the basal biological age according to Calculation formula 2 below when the subject is a female, and the biological age may be the basal biological age calculated according to Calculation formulas 1 and 2 below,

Basal biological age=M1+M2*WHR+M3*CCr+M4*Cr+M5*FEV1+M6*HbA1C+M7*AGR+M8*BUN+M9*WC+M10*TP+M11*Amylase+M12*HOMO+M13*ALT+M14*FBS+M15*SBP+M16*CPK+M17*FVC+M18*AST+M19*GGTP+M20*USG+M21*BF %+M22*BMI+M23*ALP+M24*DB+M25*ALB+M26*LBM %+M27*TC+M28*TG (Calculation formula 1)

Basal biological age=F1+F2*WHR+F3*CCr+F4*Cr+F5*FEV1+F6*HbA1c+F7*HOMO+F8*SBP+F9*BUN+F10*AST+F11*BF %+F12*TP+F13*CPK+F14*LDL+F15*AGR+F16*FVC+F17*LDH+F18*WC+F19*USG+F20*TG+F21*FBS+F22*BMI+F23*ALB+F24*PP+F25*ALP+F26*UPH+F27*DB+F28*Amylase+F29*TB+F30*HDL+F31*ALT+F32*LBM %, (Calculation formula 2)

(in this case, M1 and F1 are constants, and M2 to M28 and F2 to F32 are correlation coefficient values obtained by multiple regression analysis of a relationship between each biomarker and nominal age of the subject).

In the system, the biological age measurement unit may be configured to calculate a corrected biological age obtained by correcting the calculated basal biological age by additionally including the questionnaire information of the subject, the questionnaire information may include information about family history, smoking, drinking, and exercise, the corrected biological age may be calculated by Calculation formula 3 below, and the cancer incidence risk prediction unit may be configured to predict the risk of incidence for individual cancers based on the corrected biological age of the subject,

Corrected biological age=basal biological age+(a+b1*family history+b2*smoking+b3*drinking+b4*exercise) (Calculation formula 3)

(the family history is information about presence or absence of a family history of cancer, smoking is information about YES or NO status about smoking and pack year, drinking is information about YES or NO status about drinking and an amount of alcohol drinking per day, exercise is information about an amount of exercise per week, a is a constant obtained through regression analysis between a difference between the biological age and the nominal age and family history, smoking, drinking, and exercise information, b1 to b4 are correlation coefficient values, and b1 to b4 are correlation coefficients obtained by performing regression analysis on a correlation between the difference between the biological age before correction and the nominal age, and family history, smoking, drinking, and exercise information).

In the system, the cancer incidence risk prediction unit may be configured to, when the subject is a male, calculate a risk of individual cancer of at least one or more of risks of oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, prostate cancer, kidney cancer, bladder cancer, brain cancer, thyroid cancer, lymphoma, myeloma, and leukemia incidence, and, when the subject is a female, calculate the risk of individual cancer of at least one or more of risks of oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, breast cancer, uterine cancer, ovarian cancer, kidney cancer, bladder cancer, brain cancer, thyroid cancer, lymphoma, myeloma, and leukemia incidence, the risk of individual cancer may be calculated by multiplying the biological age of the subject by a value of relative risk of individual cancer incidence, the value of relative risk of individual cancer incidence may be a value of relative risk calculated by statistically analyzing risks of individual cancer incidence per 1 year of biological age, and the biological age may be the basal biological age or the corrected biological age.

The calculating of the risk of individual cancer incidence may calculate the risk of incidence for individual cancers based on the corrected biological age

The present invention also includes a computer server for performing a method for calculating a risk of cancer incidence based on biological age and a service server for transmitting a calculated risk of cancer incidence based on biological age via a communication network, and provides a recording medium loaded with the computer program.

The present disclosure provides the effect that a customer can easily understand his/her aging level by measuring more accurate actual age of the body compared to resident registration age, that is, biological age, using biomarkers that change according to age and can recognize his/her relative risk of cancer incidence compared to the same age according to the biological age obtained by reflecting family history and lifestyle.

The present disclosure provides the effect that can help in aging and cancer prevention management by making it easy to understand his/her own biological age and a risk level of cancer incidence, as well as enabling the maintenance and management of correct eating habits and lifestyle accordingly.

This can be used for the application of a health care field in the general local community or for providing a customized management service to the customer, and can be used for the provision of a product and content through WEB or APP of a method of predicting the risk of cancer incidence, thereby capable of further maximizing the effect.

### BRIEF DESCRIPTION OF THE DRAWINGS

FIG. 1 is a schematic configuration diagram of a method and system for predicting a risk of individual cancer incidence based on biological age obtained by reflecting family history and lifestyle according to an embodiment of the present disclosure.

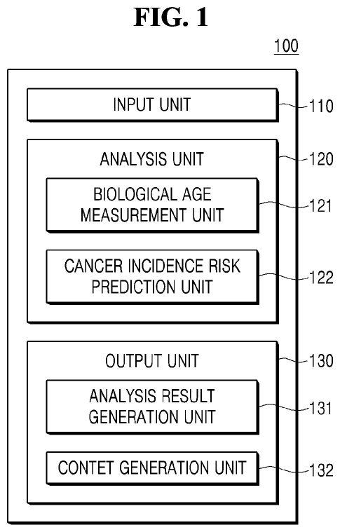

FIG. 2 is a schematic flowchart of the method and system for predicting the risk of individual cancer incidence based on biological age obtained by reflecting family history and lifestyle according to an embodiment of the present disclosure.

### DETAILED DESCRIPTION OF EMBODIMENTS

Hereinafter, embodiments of the present disclosure will be described in detail with reference to the accompanying drawings so that those of ordinary skill in the art to which the present disclosure belongs can easily carry out the present disclosure. However, the present disclosure may be implemented in various different forms and is not limited to the embodiments described herein. In order to clearly describe the present disclosure in the drawings, parts irrelevant to the description are omitted, and similar reference numerals are assigned to similar parts throughout the specification.

Hereinafter, with reference to the drawings, a method of measuring biological age and a method of predicting the risk of individual cancer incidence based on biological age that are applicable to the method and system for predicting the risk of individual cancer incidence based on biological age obtained by reflecting family history and lifestyle according to the present disclosure will be described in detail.

Meanwhile, in the present disclosure, age, actual age, nominal age, resident registration age, and legal age are expressions used with substantially the same meaning, and are the age calculated based on the time at which a person is born. In contrast, bio-age/biological age, unlike the actual age and the nominal age described above, is the age calculated according to an age calculation method of the present disclosure, and the biological age may be calculated differently even for a person of the same actual age depending on a health status and an aging status.

1. Method of Measuring Biological Age According to the Present Disclosure

1.1. Basic Information Input Step (200)

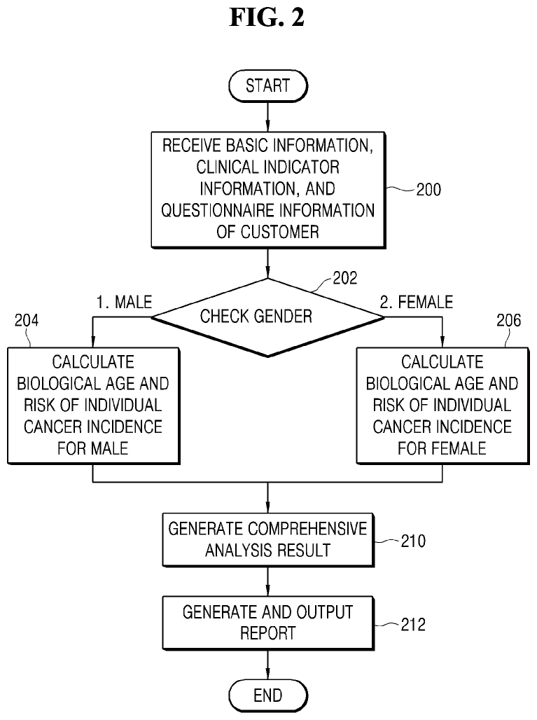

As illustrated in FIG. 2, in the basic information input step 200, \textcircled{1} basic information of a customer, \textcircled{2} biomarker information of the customer, and \textcircled{3} questionnaire information of the customer are received in order to predict the risk of individual cancer incidence based on biological age obtained by reflecting family history and lifestyle of the customer from a user through an input device of the user. This is done in an input unit 110 of a system 100 of the present disclosure, and the input device of the user may include a device such as a computer terminal or mobile terminal of the user, and a WEB or API server of a service provider connected through an APP in the device. Alternatively, in the basic information input step 200, a configuration in which information is received from an input terminal (not illustrated) of the system 100 of the present disclosure may be adopted.

The basic information of the customer includes a name, gender, age, etc. of the the customer.

Also, the biomarker information may include body mass index (BMI), lean body mass % (LBM %), body fat % (BF %), waist circumference (WC), waist-hip ratio (WHR), systolic blood pressure (SBP), pulse pressure (PP), LDH, CPK, the total cholesterol (TC), triglyceride (TG), high-density lipoprotein cholesterol (HDL), low-density lipoprotein cholesterol (LDL), homocysteine (HOMO), forced vital capacity (FVC), forced expiratory volume in one second (FEV1), total protein (TP), albumin (ALB), albumin globulin ratio (AGR), ALP, AST (GOT), ALT (GPT), gamma GMT (GGTP), total bilirubin (TB), direct bilirubin (DB), fasting blood sugar (FBS), glycated hemoglobin (HbA1c), Amylase, the creatinine (Cr), creatinine clearance rate (CCr), blood urea nitrogen (BUN), urine specific gravity (USG), and urin pH (UPH), etc., and the questionnaire information may include YES or NO status about smoking, pack year, YES or NO status about drinking, amount of alcohol drinking per day, amount of exercise per week, cancer family history, etc.

1.2. Gender Check Step (202)

When basic information of the customer and biomarker information and questionnaire information for the customer are provided (step 200), it is checked whether gender information included in the basic information of the customer indicates a male or a female (step 202).

1.3. Biological Age and Cancer Incidence Risk Calculation Steps (204, 206)

1.3.1. Step of Calculating Biological Age and Risk of Cancer Incidence for Male (204)

When the customer indicates a male, a biological age measurement unit 121 of an analysis unit 120 of a biological age measurement system of the present disclosure measures biological age of the customer through biomarker information about the customer according to Calculation formula 1, and then calculates the risk of incidence for individual cancers using the same (step 204).

(1) Basal Biological Age Calculation Step

Using the biomarker information of the customer, basal biological age is calculated according to Calculation formula 1. In this time, the biomarker information includes the waist-hip ratio (WHR), creatinine clearance rate (CCr), creatinine (Cr), forced expiratory volume in one second (FEV1), glycated hemoglobin (HbA1c), albumin globulin ratio (AGR), blood urea nitrogen (BUN), waist circumference (WC), total protein (TP), Amylase, the homocysteine (HOMO), ALT (GPT), fasting blood sugar (FBS), systolic blood pressure (SBP), CPK, the forced vital capacity (FVC), AST (GOT), gamma GTP (GGTP), urine specific gravity (USG), body fat % (BF %), body mass index (BMI), ALP, the direct bilirubin (DB), albumin (ALB), lean body mass % (LBM %), total cholesterol (TC), and triglyceride (TG).

Basal biological age=M1+M2*WHR+M3*CCr+M4*Cr+M5*FEV1+M6*HbA1C+M7*AGR+M8*BUN+M9*WC+M10*TP+M11*Amylase+M12*HOMO+M13*ALT+M14*FBS+M15*SBP+M16*CPK+M17*FVC+M18*AST+M19*GGTP+M20*USG+M21*BF %+M22*BMI+M23*ALP+M24*DB+M25*ALB+M26*LBM %+M27*TC+M28*TG (Calculation formula 1)

Calculation formula 1 is obtained by multiplying a different correlation coefficient for each of the biomarker information and then adding the multiplication results.

In Calculation formula 1 above, M1 is a constant, and M2 to M28 are preset values, are correlation coefficient values between biomarkers and nominal age (resident registration age) of a subject and the nominal age, that are obtained by performing multiple regression analysis on the nominal age of the subject and biomarkers of the subject from predetermined sample data, and may have negative values or positive values according to biomarker information.

The M1 obtained from the sample data applied in the present disclosure is a constant, M2, M6, M8, M11, M12, M14, M15, M18, M21, M25 have positive values, and the remaining M3, M4, M5, M7, M9, M10, M13, M16, M17, M19, M20, M22, M23, M24, M26, M27, and M28 have negative values.

That is, the correlation coefficients M2 to M28 are correlation coefficients between the nominal age of and biomarkers of the subjects constituting the sample data. Among the indicators obtained from the sample data applied in the present disclosure, indicators FEV1 (forced expiratory volume in one second) and FVC (forced vital capacity) were found to have the highest correlation with age for a male (r=−0.5436 and −0.4812; p<0.0001).

Meanwhile, the present disclosure does not have specificity in sample data, and the sample data referred to in the present disclosure is sufficient as long as it is possible to obtain each piece of of the biomarker information of Calculation formula 1 and perform regression analysis to obtain each of the correlation coefficients to a predetermined degree of statistical significance.

(2) Biological Age Correction Step for Calculating Risk of Cancer Incidence

In order to calculate the risk of cancer incidence, the basal biological age calculated by Calculation formula 1 above can be corrected by Calculation formula 2 to be described later.

In the correction of biological age, corrected biological age is calculated by using the questionnaire information such as YES or NO status about smoking, pack year, YES or NO status about drinking, amount of alcohol drinking per day, amount of exercise per week, and cancer family history as variables for the calculated basal biological age.

Corrected biological age=basal biological age+(a+b1*family history+b2*smoking+b3*drinking+b4*exercise) <Calculation formula 2>

a is a constant obtained through regression analysis between a difference between biological age and nominal age and family history, smoking, drinking, and exercise information, b1 to b4 are correlation coefficient values, and family history is information about YES or NO of a cancer family history, smoking is information about YES or NO status about smoking and pack year, drinking is information about YES or NO status about drinking and an amount of alcohol drinking per day, and exercise is information about an amount of exercise per week.

The unit amount of these pieces of information need not be limited to a specific unit amount. For example, in the case of smoking information, the amount of tobacco consumed per day and the number of years of smoking may be included. Alcohol drinking may be replaced with information such as the number of drinking times per week and amount of alcohol consumed per drink, and information about exercise may be replaced with information such as, the number of times of exercise per week and exercise intensity high/middle/low in one exercise. A more detailed example of calculation is described below.

The preset values a, and b1 to b4 are a constant and correlation coefficient values generated through multiple regression analysis from sample data according to Calculation formula 3 below, respectively. That is, a, and b1 to b4 may be a constant and correlation coefficient values obtained by performing multiple regression analysis on (basal biological age−nominal age), family history, smoking, drinking, and exercise information in the sample data, in which family history, smoking, drinking, exercise information, basal biological age, and nominal age are known, as shown in Calculation formula 3 below, respectively.

Basal biological age−nominal age=a+b1*family history+b2*smoking+b3*drinking+b4*exercise <Calculation formula 3>

The YES or NO status about smoking described above is classified according to whether or not the person has smoked more than 5 packs (100 cigarettes) in his/her lifetime, and pack year (amount of cigarettes smoked per day and number of years smoked. pack and year) is calculated as the number of pack-years before quitting smoking if the person smoked in the past but does not smoke now, and if the person is still smoking, the pack year is calculated as “pack-year=(pack/day)×years”. The YES or NO status about drinking and amount of alcohol drinking per day are calculated using “Alcohol intake per day g/day=frequency of intake x intake per drink x alcohol content of soju 22%×0.8 g” according to an alcohol content calculation formula “[Intake (mL)×Alcohol content (%)×0.8 (Alcohol specific gravity)]/100” to determine how many days a week a person drinks on average and how much a person drinks per day when drinking (regardless of the type of alcohol, 1 can of beer (355 cc) equals 1.6 glasses of beer), and are classified according to the WHO daily alcohol standard. Also, in the case of exercise, the amount of activity for one week is calculated as “total activity for one week=Y each physical activity MET×MIN”, and each physical activity is divided into strenuous physical activity, moderate-intensity physical activity, and walking, where the strenuous physical activity may be calculated as “number of times per week×8.0 MET×60 minutes”, the moderate-intensity physical activity as “number of times per week×4.0 MET×60 minutes”, and the walking as “number of times per week×3.3 MET×30 minutes”.

(3) Step of Predicting Risk of Individual Cancer Incidence Based on Biological Age

A cancer incidence risk prediction unit 122 of the analysis unit 120 calculates the risk of individual cancer incidence of the customer through the biological age measured above, according to Calculation formula 4. Here, the individual cancers may include oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, prostate cancer, kidney cancer, bladder cancer, brain cancer, thyroid cancer, lymphoma, myeloma, and leukemia.

Risk of individual cancer incidence=Ci*biological age <Calculation formula 4>

In Calculation formula 4 above, the individual cancers include oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, prostate cancer, kidney cancer, bladder cancer, brain cancer, thyroid cancer, lymphoma, myeloma, and leukemia, and Ci is a value of relative risk for each individual cancer.

Calculation formula 4 can be expressed as follows for each individual cancer.

Risk of oral cancer incidence=C1*biological age

Risk of pharyngeal cancer incidence=C2*biological age

Risk of esophageal cancer incidence=C3*biological age

Risk of stomach cancer incidence=C4*biological age

Risk of small intestine cancer incidence=C5*biological age

Risk of colorectal cancer incidence=C6*biological age

Risk of liver cancer incidence=C7*biological age

Risk of gallbladder cancer incidence=C8*biological age

Risk of pancreatic cancer incidence=C9*biological age

Risk of laryngeal cancer incidence=C10*biological age

Risk of lung cancer incidence=C11*biological age

Risk of skin cancer incidence=C12*biological age

Risk of prostate cancer incidence=C13*biological age

Risk of kidney cancer incidence=C14*biological age

Risk of bladder cancer incidence=C15*biological age

Risk of brain cancer incidence=C16*biological age

Risk of thyroid cancer incidence=C17*biological age

Risk of lymphoma incidence=C18*Biological age

Risk of myeloma incidence=C19*biological age

Risk of leukemia incidence=C20*biological age

That is, in Calculation formula 4, the risk of oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, prostate cancer, kidney cancer, bladder cancer, brain cancer, thyroid cancer, lymphoma, myeloma, and leukemia is calculated by multiplying the risk of each individual cancer incidence per 1 year of biological age.

The calculation of the risk of individual cancer incidence is calculated by multiplying the biological age of the subject by the relative risk of individual cancer incidence, and in Calculation formula 4, the individual cancer incidence correlation coefficient (risk of individual cancer incidence Ci: C1 to C20) is a preset value, is values of the relative risk obtained by statistically analyzing the risk of each individual cancer incidence per 1 year of biological age for a number of men prepared in advance for the present disclosure, and has a value less than or greater than 1 depending on the biological age. That is, in Calculation formula 4, C1 to C20 all have a value greater than 1.

In Calculation formula 4, the biological age may be the basal biological age or corrected biological age calculated above, and the method of calculating the values of relative risk of individual cancer incidence will be described in Sections 1.3.3 below

1.3.2. Step of Calculating Biological Age and Risk of Cancer Incidence for Female (206)

When the gender information included in the basic information of the customer above indicates a female, the biological age measurement unit 121 of the analysis unit 120 measures biological age of the customer through biomarker information about the customer according to Calculation formula 5, and then calculates the risk of incidence for individual cancers using the same (step 206).

(1) Basal Biological Age Calculation Step

Using the biomarker information of the female customer, the basal biological age is calculated according to Calculation formula 5 below. Here, the biomarker information includes the waist-hip ratio (WHR), creatinine clearance rate (CCr), creatinine (Cr), forced expiratory volume in one second (FEV1), glycated hemoglobin (HbA1c), homocysteine (HOMO), systolic blood pressure (SBP), blood urea nitrogen (BUN), AST (GOT), body fat % (BF %), total protein (TP), CPK, the low-density lipoprotein cholesterol (LDL), albumin globulin ratio (AGR), forced vital capacity (FVC), LDH, the waist circumference (WC), urine specific gravity (USG), triglyceride (TG), fasting blood sugar (FBS), body mass index (BMI), albumin (ALB), pulse pressure (PP), ALP (ALP), urin pH (UPH), direct bilirubin (DB), Amylase, the total bilirubin (TB), high-density lipoprotein cholesterol (HDL), ALT (GPT), and lean body mass % (LBM %).

Basal biological age=F1+F2*WHR+F3*CCr+F4*Cr+F5*FEV1+F6*HbA1c+F7*HOMO+F8*SBP+F9*BUN+F10*AST+F11*BF %+F12*TP+F13*CPK+F14*LDL+F15*AGR+F16*FVC+F17*LDH+F18*WC+F19*USG+F20*TG+F21*FBS+F22*BMI+F23*ALB+F24*PP+F25*ALP+F26*UPH+F27*DB+F28*Amylase+F29*TB+F30*HDL+F31*ALT+F32*LBM % <Calculation formula 5>

Calculation formula 5 is obtained by multiplying a different correlation coefficient for each of the biomarker information and then adding the multiplication results. In Calculation formula 5, F1 is a constant, and F2 to F32 are preset values, are correlation coefficients obtained by analyzing biomarker information for a female for the present disclosure (like the correlation coefficients in Calculation formula 1, correlation coefficients obtained by performing multiple regression analysis. correlation coefficients by setting biomarkers as independent variables and nominal age as dependent variables, from the sample data of sample subjects whose nominal age and biomarkers are known), and may have a negative value or a positive value according to the biomarker information.

The F1 obtained from the sample data applied in the present disclosure is a constant, F2, F6, F7, F8, F9, F10, F11, F13, F14, F17, F20, F21, F23, F24, F26, F28, F29 have positive values, and the remaining F3, F4, F5, F12, F15, F16, F18, F19, F22, F25, F27, F30, F31, and F32 have negative values.

According to the basal biological age calculation formula according to the present disclosure, according to the correlation coefficient obtained as above, for example, the waist-hip ratio (WHR) of the customer can be interpreted as increasing the biological age by a value multiplied by M2 for a male and increasing the biological age by a value multiplied by F2 for a female, and the creatinine clearance rate (CCr) of the customer can be interpreted as decreasing the biological age by a value multiplied by M3 for a male and decreasing the biological age by a value multiplied by F3 for a female.

The correlation coefficients are correlation coefficients between age and clinical parameters, and for a female, the indicators most highly correlated with age were the forced expiratory volume in one second (FEV1) and the forced vital capacity (FVC) (r=−0.4422 and −0.3905; p<0.0001), as in men, and the waist-hip ratio (WHR) also showed a high correlation (r=0.4225; p<0.0001). The method of obtaining the correlation coefficients is the same as the method of obtaining the correlation coefficient for a male.

(2) Biological Age Correction Step

Even in the case of a female, a corrected biological age may be calculated by correcting basal biological age according to Calculation formulas 2 and 3 described above by using the questionnaire information such as YES or NO status about smoking, pack year, YES or NO status about drinking, amount of alcohol drinking per day, amount of exercise per week, and cancer family history as variables for the calculated basal biological age (step 206).

According to the relational formula of Calculation formula 3, the more the person have the family history, smoking, or drinking, the more the basal biological age of the person increases, and the more the person exercise, the more the basal biological age of the person decreases, and the corrected biological age obtained by reflecting the family history and major lifestyle is calculated according to Calculation formula 2 (step 206).

(3) Step of Predicting Risk of Individual Cancer Incidence Based on Biological Age

Even in the case of a female customer, the cancer incidence risk prediction unit 122 of the analysis unit 120 measures the risk of individual cancer incidence of the customer through the biological age measured above according to Calculation formula 6 (step 206). Here, the individual cancers may include a total of 22 types of oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, breast cancer, uterine cancer, ovarian cancer, kidney cancer, bladder cancer, brain cancer, thyroid cancer, lymphoma, myeloma, and leukemia.

Risk of individual cancer incidence=Di*biological age <Calculation formula 6>

In Calculation formula 6, individual cancers include oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, breast cancer, uterine cancer, ovarian cancer, kidney cancer, bladder cancer, brain cancer, thyroid cancer, lymphoma, myeloma, and leukemia, and Di is a value of relative risk for each individual cancer.

Calculation formula 6 can be expressed as follows for each individual cancer.

Risk of oral cancer incidence=D1*biological age

Risk of pharyngeal cancer incidence=D2*biological age

Risk of esophageal cancer incidence=D3*biological age

Risk of stomach cancer incidence=D4*biological age

Risk of small intestine cancer incidence=D5*biological age

Risk of colorectal cancer incidence=D6*biological age

Risk of liver cancer incidence=D7*biological age

Risk of gallbladder cancer incidence=D8*biological age

Risk of pancreatic cancer incidence=D9*biological age

Risk of laryngeal cancer incidence=D10*biological age

Risk of lung cancer incidence=D11*biological age

Risk of skin cancer incidence=D12*biological age

Risk of breast cancer incidence=D13*biological age

Risk of uterine cancer incidence=D14*biological age

Risk of ovarian cancer incidence=D15*biological age

Risk of kidney cancer incidence=D16*biological age

Risk of bladder cancer incidence=D17*biological age

Risk of brain cancer incidence=D18*biological age

Risk of thyroid cancer incidence=D19*biological age

Risk of lymphoma incidence=D20*Biological age

Risk of myeloma incidence=D21*biological age

Risk of leukemia incidence=D22*biological age

That is, in Calculation formula 6, the risk of oral cancer, pharyngeal cancer, esophageal cancer, stomach cancer, small intestine cancer, colorectal cancer, liver cancer, gallbladder cancer, breast cancer, uterine cancer, ovarian cancer, laryngeal cancer, lung cancer, skin cancer, prostate cancer, kidney cancer, bladder cancer, brain cancer, thyroid cancer, lymphoma, myeloma, and leukemia is calculated by multiplying the risk of each individual cancer incidence per 1 year of biological age.

The calculation of the risk of individual cancer incidence is calculated by multiplying the biological age of the subject by the relative risk of individual cancer incidence, and in Calculation formula 6, the individual cancer incidence correlation coefficient (risk of individual cancer incidence Ci: C1 to C20) is a preset value, is values of the relative risk obtained by statistically analyzing the risk of each individual cancer incidence per 1 year of biological age for a number of women prepared in advance for the present disclosure, and has a value less than or greater than 1 depending on the biological age. That is, in Calculation formula 6, D1 to D22 all have a value greater than 1.

In Calculation formula 6, the biological age may be the basal biological age or corrected biological age calculated above.

1.3.3. Method of Calculating Relative Risk of Individual Cancer Incidence

The relative risk of individual cancer incidence, which is each of the coefficient values C1 to C20 and D1 to D22 in Calculation formula 4 and Calculation formula 6, was calculated using the Cox proportional hazards model.

The Cox proportional hazards model is a model built on the assumption that there is a log-linear relationship between a survival function and a variable. According to this, when xᵢ=(xᵢ₂, . . . , xᵢₚ) is a variable for iₜₕ member, the survival function S(t) is expressed as a time t and a variable xᵢ as shown in expression \textcircled{1} below.

If this is applied to the present disclosure, when predetermined sample data having individual cancer onset data is substituted into the Cox proportional hazards model expression \textcircled{1} below, a model parameter b1 is calculated, and according to a relative risk calculation formula in Expression \textcircled{2} below, the relative risk e^{b1} when biological age increases from k to (k+1) by one year is obtained.

Cox Proportional Hazards Model:

S(t|xᵢ)=S₀(t)exp(b₁xᵢ₁+ . . . +bₚxᵢₚ) \textcircled{1}

Relative risk:

$$\frac{\frac{S ( t \vert {x}_{1} = k + 1 )}{{S}_{0} ( t )}}{\frac{S ( t \vert {x}_{1} = k )}{{S}_{0} ( t )}} = \frac{S ( t \vert {x}_{1} = k + 1 )}{S ( t \vert {x}_{1} = k )} = \frac{{e}^{{b}_{1} ( k + 1 ) + {b}_{2} {x}_{2} + … + {b}_{p} {x}_{p}}}{{e}^{{b}_{1} ( k ) + {b}_{2} {x}_{2} + … + {b}_{p} {x}_{p}}} = {e}^{{b}_{1}}$$

For example, when explaining a process of calculating the risk of lung cancer incidence per year increase in biological age in a female group, when an explanatory variable is biological age, the lung cancer incidence over time is given as S(t)=S₀(t)exp(b₁xᵢ₁) from the Cox proportional hazards model, (xii is biological age of i-th sample data), and when a regression coefficient b1 is calculated, the relative risk of lung cancer incidence per year increase in biological age is calculated from Equation (2), from which the relative risk of lung cancer incidence D11=e^{b1} is obtained.

The Cox proportional hazards model is a type of survival analysis. Survival analysis refers to an analysis method that analyzes the time it takes for an event such as death or disease to occur. In the present disclosure, the effect of various risk factors such as biological age, smoking history, alcohol drinking history, cancer family history, and exercise amount on survival (the period from the time of participation in the study to the cancer incidence) is investigated. Therefore, the Cox proportional hazards model is a function value calculated according to the numerical values of various risk factors, and the comparative risk is a value calculated to see the effect of the risk factor to be checked among risk factor information of the Cox proportional hazards model on survival. For example, in the case of smoking compared to non-smoking, the relative risk in survival can be obtained by dividing the Cox proportional hazards model in smokers by the Cox proportional hazards model in non-smokers.

In the present disclosure, the calculation of the regression coefficient may be calculated in a regression analysis process through a Cox regression analysis method according to a conventional Cox proportional hazards model. In the embodiment of the present disclosure, SAS 9.4 version was used, and data obtained from a follow-up survey of approximately 160,000 people at Yonsei University Medical Center for approximately 18 years from 2002 was used as sample data. Meanwhile, it will be obvious to those skilled in the art that the technical idea of the present disclosure is not dependent on specific software or the specific sample data for performing the Cox regression analysis.

1.4. Report Generation and Output Step

As described above, when biological age measurement and risk of individual cancer incidence measurement based on biological age are completed, an output unit 130 generates a comprehensive analysis result for prediction of the risk of individual cancer incidence based on the biological age including biological age analysis of the customer (step 210). The result of the comprehensive analysis includes actual age and measured biological age of the customer, result of the risk of each individual cancer incidence, and statistical information about cancer incidence of the same sex and the same age for each individual cancer. Here, the result of the comprehensive analysis is divided into five grades, such as good, caution, warning, risk, and high risk, according to the risk of individual cancer incidence of the customer, and includes a pre-determined prevention practice guide corresponding to each result.

When the comprehensive analysis result described above is generated, the output unit 130 generates biological age and report information of the risk of individual cancer incidence based on biological age (step 210), and outputs the report information in a form that can be provided to the customer (step 212).

Outputting the report information in the form that can be provided to the customer includes displaying the report information on a screen, providing the report information in a printable file format, or providing the report information in a form of an API.

It is obvious to those skilled in the art that the procedure for calculating the risk of cancer incidence based on biological age described above can be implemented and carried out through a computer program. The present disclosure includes a computer program and a recording medium onto which the program is loaded, and a computer device and server which are loaded with the program and perform the procedures described above.

The present disclosure also includes the system and service server for calculating the risk of cancer incidence for a subject by performing the method and procedure described above and transmitting the risk of cancer incidence to a terminal device possessed by the subject through a communication network.

Although the cancer risks prediction method and system based on biological age using family history and lifestyles information have been described with reference to the specific embodiments, they are not limited thereto. Therefore, it will be readily understood by those skilled in the art that various modifications and changes can be made thereto without departing from the spirit and scope of the present invention defined by the appended claims.

1. A system for calculating a risk of cancer incidence based on biological age, which is applied to a system for predicting a risk of individual cancer incidence, comprising:
an input unit configured to receive basic information such as gender and age, biomarker information including a medical check-up result, and questionnaire information of a subject; and
an analysis unit that comprises a biological age measurement unit configured to calculate biological age of the subject based on the basic information and the biomarker information of the subject, and a cancer incidence risk prediction unit configured to predict a risk of incidence for individual cancers based on the biological age of the subject.

2. The system for claim 1, wherein
the biomarker information includes at least one or more of waist-hip ratio (WHR), creatinine clearance rate (CCr), creatinine (Cr), forced expiratory volume in one second (FEV1), glycated hemoglobin (HbA1c), albumin globulin ratio (AGR), blood urea nitrogen (BUN), waist circumference (WC), total protein (TP), Amylase, homocysteine (HOMO), ALT (GPT), fasting blood sugar (FBS), systolic blood pressure (SBP), CPK, forced vital capacity (FVC), AST (GOT), gamma GTP (GGTP), urine specific gravity (USG), body fat % (BF %), body mass index (BMI), ALP, direct bilirubin (DB), albumin (ALB), lean body mass % (LBM %), total cholesterol (TC), and triglyceride (TG),
the biological age measurement unit is configured to calculate basal biological age according to Calculation formula 1 below when the subject is a male and calculate the basal biological age according to Calculation formula 2 below when the subject is a female, and
the biological age is the basal biological age calculated according to Calculation formulas 1 and 2 below,
Basal biological age=M1+M2*WHR+M3*CCr+M4*Cr+M5*FEV1+M6*HbA1C+M7*AGR+M8*BUN+M9*WC+M10*TP+M11*Amylase+M12*HOMO+M13*ALT+M14*FBS+M15*SBP+M16*CPK+M17*FVC+M18*AST+M19*GGTP+M20*USG+M21*BF %+M22*BMI+M23*ALP+M24*DB+M25*ALB+M26*LBM %+M27*TC+M28*TG, (Calculation formula 1)
Basal biological age=F1+F2*WHR+F3*CCr+F4*Cr+F5*FEV1+F6*HbA1c+F7*HOMO+F8*SBP+F9*BUN+F10*AST+F11*BF %+F12*TP+F13*CPK+F14*LDL+F15*AGR+F16*FVC+F17*LDH+F18*WC+F19*USG+F20*TG+F21*FBS+F22*BMI+F23*ALB+F24*PP+F25*ALP+F26*UPH+F27*DB+F28*Amylase+F29*TB+F30*HDL+F31*ALT+F32*LBM % (Calculation formula 2)
(in this case, M1 and F1 are constants, and M2 to M28 and F2 to F32 are correlation coefficient values obtained by multiple regression analysis of a relationship between each biomarker and nominal age of the subject).

3. The system for claim 2, wherein
the biological age measurement unit is configured to calculate a corrected biological age obtained by correcting the calculated basal biological age by additionally including the questionnaire information of the subject,
the questionnaire information includes information about family history, smoking, drinking, and exercise,
the corrected biological age is calculated by Calculation formula 3 below, and
the cancer incidence risk prediction unit is configured to predict the risk of incidence for individual cancers based on the corrected biological age of the subject,
Corrected biological age=basal biological age+(a+b1*family history+b2*smoking+b3*drinking+b4*exercise) (Calculation formula 3)
(the family history is information about presence or absence of a family history of cancer, smoking is information about YES or NO status about smoking and pack year, drinking is information about YES or NO status about drinking and an amount of alcohol drinking per day, exercise is information about an amount of exercise per week, a is a constant obtained through regression analysis between a difference between the biological age and the nominal age and family history, smoking, drinking, and exercise information, b1 to b4 are correlation coefficient values, and b1 to b4 are correlation coefficients obtained by performing regression analysis on a correlation between the difference between the biological age before correction and the nominal age, and family history, smoking, drinking, and exercise information).

4. The system for claim 3, wherein
the cancer incidence risk prediction unit is configured to
when the subject is a male, calculate a risk of individual cancer of at least one or more of risks of oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, prostate cancer, kidney cancer, bladder cancer, brain cancer, thyroid cancer, lymphoma, myeloma, and leukemia incidence, and
when the subject is a female, calculate the risk of individual cancer of at least one or more of risks of oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, breast cancer, uterine cancer, ovarian cancer, kidney cancer, bladder cancer, brain cancer, thyroid cancer, lymphoma, myeloma, and leukemia incidence,
the risk of individual cancer is calculated by multiplying the biological age of the subject by a value of relative risk of individual cancer incidence,
the value of relative risk of individual cancer incidence is a value of relative risk calculated by statistically analyzing risks of individual cancer incidence per 1 year of biological age, and
the biological age is the basal biological age or the corrected biological age.

5. The system for claim 1, wherein
the biological age measurement unit is configured to calculate the biological age of the subject based on the basic information, biomarkers, and questionnaire information of the subject,
the questionnaire information includes information about family history, smoking, drinking, and exercise, and
the cancer incidence risk prediction unit is configured to predict the risk of incidence for individual cancers based on the biological age

6. A method of calculating a risk of cancer incidence based on biological age, comprising:
receiving basic information such as gender and age, biomarker information including a medical check-up result, and questionnaire information of a cancer incidence risk calculation subject;
checking gender information of the subject based on the information input in the receiving of basic information;
calculating biological age of the subject based on the biomarker information; and
calculating a risk of individual cancer incidence for the subject based on the calculated biological age.

7. The method of claim 6, wherein
the biomarker information includes at least one or more of waist-hip ratio (WHR), creatinine clearance rate (CCr), creatinine (Cr), forced expiratory volume in one second (FEV1), glycated hemoglobin (HbA1c), albumin globulin ratio (AGR), blood urea nitrogen (BUN), waist circumference (WC), total protein (TP), Amylase, homocysteine (HOMO), ALT (GPT), fasting blood sugar (FBS), systolic blood pressure (SBP), CPK, forced vital capacity (FVC), AST (GOT), gamma GTP (GGTP), urine specific gravity (USG), body fat % (BF %), body mass index (BMI), ALP, direct bilirubin (DB), albumin (ALB), lean body mass % (LBM %), total cholesterol (TC), and triglyceride (TG), and
the biological age measurement unit is configured to calculate the biological age according to calculation formula 1 below when the subject is a male and calculate the biological age according to calculation formula 2 below when the subject is a female,
Biological age=M1+M2*WHR+M3*CCr+M4*Cr+M5*FEV1+M6*HbA1C+M7*AGR+M8*BUN+M9*WC+M10*TP+M11*Amylase+M12*HOMO+M13*ALT+M14*FBS+M15*SBP+M16*CPK+M17*FVC+M18*AST+M19*GGTP+M20*USG+M21*BF %+M22*BMI+M23*ALP+M24*DB+M25*ALB+M26*LBM %+M27*TC+M28*TG, (Calculation formula 1)
Biological age=F1+F2*WHR+F3*CCr+F4*Cr+F5*FEV1+F6*HbA1c+F7*HOMO+F8*SBP+F9*BUN+F10*AST+F11*BF %+F12*TP+F13*CPK+F14*LDL+F15*AGR+F16*FVC+F17*LDH+F18*WC+F19*USG+F20*TG+F21*FBS+F22*BMI+F23*ALB+F24*PP+F25*ALP+F26*UPH+F27*DB+F28*Amylase+F29*TB+F30*HDL+F31*ALT+F32*LBM % (Calculation formula 2)
(in this case, M1 and F1 are constants, and M2 to M28 and F2 to F32 are correlation coefficient values obtained by multiple regression analysis of a relationship between each biomarker and nominal age of the subject).

8. The method of claim 7, wherein
the calculating of biological age further comprises
calculating a corrected biological age obtained by correcting the calculated basal biological age by additionally including the questionnaire information of the subject, and
the questionnaire information includes information about family history, smoking, drinking, and exercise, and
the corrected biological age is calculated by Calculation formula 3 below,
Corrected biological age=basal biological age+(a+b1*family history+b2*smoking+b3*drinking+b4*exercise) (Calculation formula 3)
(the family history is information about presence or absence of a family history of cancer, smoking is information about YES or NO status about smoking and pack year, drinking is information about YES or NO status about drinking and an amount of alcohol drinking per day, exercise is information about an amount of exercise per week, a is a constant obtained through regression analysis between a difference between biological age and nominal age and family history, smoking, drinking, and exercise information, and b1 to b4 are correlation coefficient values).

9. The method of claim 8, wherein
in the calculating of the risk of individual cancer incidence,
when the subject is a male, the risk of individual cancer incidence of at least one or more of risks of oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, prostate cancer, kidney cancer, bladder cancer, brain cancer, thyroid cancer, lymphoma, myeloma and leukemia incidence is calculated, and
when the subject is a female, the risk of individual cancer incidence of at least one or more of risks of oral cancer, pharyngeal cancer, esophageal cancer, stomach cancer, small intestine cancer, colorectal cancer, liver cancer, gallbladder cancer, pancreatic cancer, laryngeal cancer, lung cancer, skin cancer, breast cancer, uterine cancer, ovarian cancer, kidney cancer, bladder cancer, brain cancer, thyroid cancer, lymphoma, myeloma, and leukemia incidence is calculated,
the risk of individual cancer incidence is calculated by multiplying the biological age of the subject by a value of relative risk of individual cancer incidence, and
the value of relative risk of individual cancer incidence is a value of relative risk calculated by statistically analyzing risks of individual cancer incidence per 1 year of biological age.

10. The method of claim 9, wherein
in the calculating of the risk of individual cancer incidence, the risk of incidence for individual cancers is calculated based on the corrected biological age.

11. A recording medium loaded with a computer program for performing the method of calculating a risk of cancer incidence based on biological age according to claim 6.

12. A computer server loaded with a computer program for performing the method of calculating a risk of cancer incidence based on biological age according to claim 6.

13. A service server for transmitting a risk of cancer incidence based on biological age calculated through the method of calculating the risk of cancer incidence based on biological age according to claim 6 to the subject through a communication network.
